(19) United States
(12) Patent Application Publication
(10) Pub. No.: US 2009/0203789 A1
(43) Pub. Date: Aug. 13, 2009

(54) Fast Dissolution Amino Acid Composition

(75) Inventors: Ken Clement, Oakville (CA); Jason Peters, Oakville (CA); Marvin A. Heuer, Oakville (CA); Shan Chaudhuri, Brampton (CA)

Correspondence Address: TORYS LLP, 79 WELLINGTON STREET WEST, SUITE 3000, BOX 270, TD CENTRE, TORONTO, ON, M5K 1N2, CA

(73) Assignee: MULTI FORMULATIONS LTD., Oakville (CA)

(21) Appl. No.: 12/023,289

(22) Filed: Jan. 31, 2008

(51) Int. Cl. A61K 31/195; A61P 43/00

(52) U.S. Cl. 514/561; 514/565; 514/567

(57) ABSTRACT

The present invention relates to oral dosage formulations for the rapid release of amino acids. A rapid release is achieved through the process of milling whereby the milled amino acids are more readily bioavailable.

### RELATED APPLICATIONS

The present application is related to U.S. patent application Ser. No. 11/709,525 and U.S. patent application Ser. No. 11/709,526, both of which claim priority from U.S. Provisional Patent Application No. 60/776,325.

### FIELD OF THE INVENTION

The present invention relates generally to a rapid release characterized by an increase in the immediacy of absorption of an oral formulation of a dietary supplement. More particularly, the invention relates to rapid release and increased rate of bioavailability of an oral formulation of amino acids.

### BACKGROUND OF THE INVENTION

Poorly-soluble compounds are described as either sparingly soluble or insoluble in polar or non polar solvents depending on the hydrophilicity of lipophilicity of said compounds. Many compounds, particularly in the dietary supplement industry, fall into the class of low solubility. This not only presents a problem in terms of bioavailability but also in terms of reducing or preventing toxicity and irregular absorption in the intestinal tract. Therefore, it is a challenge to make these compounds, which will be used in a biological system, e.g., orally ingested by a human, such that they will be more readily bioavailable.

Various methods have been explored to achieve increased bioavailability within the pharmaceutical industry including chemical methods, physiological procedures, and pharmaceutical methods. However, many of these methods and techniques cannot be applied or utilized by the dietary supplement industry.

### SUMMARY OF THE INVENTION

The present invention relates to a method for increasing the immediacy of dissolution and absorption of a dietary supplement in a mammal. The desired results are obtained by providing a rapid release formulation which provides an enhanced compound transfer of the supplement to the mammal via an increase in the rate of dissolution. For the purpose of this invention, micronization techniques are employed for sizing solid compounds and increasing the immediacy of dissolution and absorption. The increase in the immediacy of dissolution and absorption will allow enhanced and quicker compound transfer to the systemic parts of the body following ingestion, and thus increase efficacy of the composition.

### DETAILED DESCRIPTION OF THE INVENTION

### Definitions

For the purposes of the present invention, the terms micronization, milling, particle-milling, nano-milling and fine-milling are used interchangeably, wherein they refer to a technology, process and end-products involved in or leading to a narrowing of particle size range and a concomitant reduction in the average particle size. For the purposes of the present invention, acceptable milled-particle sizes are in the range of from about 1 nanometer to about 500 microns. Furthermore, for the purposes of the present invention, substances that have undergone a milling process are termed milled, fine-milled, nano-milled or nano.

Although it is understood by the inventors that dietary supplements from natural sources must inherently undergo a degree of processing prior to use, as used herein the terms ‘unprocessed’ or ‘regular’ refer the physical state of ingredients or compounds, which have not been subjected to a micronization process.

As used herein, the term ‘bioavailability’ refers to the amount of a substance available at a given site of physiological activity after administration. Bioavailability of a given substance is affected by a number of factors including but not limited to degradation and absorption of that substance. Orally administered substances are subject to excretion prior to complete absorption, thereby decreasing bioavailability as compared to other administration routes.

As used herein, the term ‘molecule’ refers to the smallest size attainable of a given substance wherein the chemical properties of said substance are retained.

As used herein, the term ‘solubility’ refers to the amount of or degree to which a substance or solute will dissolve within a given solvent. Several factors affect the solubility of a given substance. These factors include but are not limited to: specific properties of the solute and the solvent, polarity of the solute, the polarity of the solvent, the temperature, and the pressure. The term ‘absolute solubility’, as used herein, refers to the solubility of a given substance under conditions in which time is not a factor, i.e. infinite time. It is understood that a substance may be in ‘suspension’ rather than solution but will appear to be in solution.

As used herein, the term ‘dissolution’ refers to the process of a solute going into solution or solubilizing. Dissolution is dependent upon several factors including but not limited to: temperature, agitation and surface area of a given particle.

### Micronization Techniques

Micronization within the scope of this invention can be taken to mean any one of a number of micronization techniques. Although specific techniques are disclosed herein, the invention is more general than any specific micronization or particle-milling technique. This includes the discovery that by placing the amino acids in a rapid release formulation, the increase in the immediacy of dissolution as compared to regular formulations provides improved and unexpected results.

Micronization or particle-milling for the purposes of the present invention is preformed by a variety of methods. Dry milling or nanosuspensions are often made by air jet milling and wet milling in pearl mills and rotor-stator mills as commonly known in the art as part of the micronization process.

Methods of milling particles can also be those such as hammer mills, cryogenic hammer mills, fluid and air jet milling, jaw crushing, and high-pressure dispersion milling. These are methods of medialess milling. Hammer milling produces particles of typically 30-500 microns. At ambient temperatures, rotating hammers which strike the particles repeatedly reduce the particle size to a point where they can pass through a screen having a given mesh size. If required, the process can also be done at lower temperatures in cases where a reduced temperature is required to fracture a given particle.

During the process of jet milling, particles are suspended in flowing streams of air where they are targeted at either themselves or a stationary target. This results in a fine grind with a particle size of typically 1-10 microns being produced.

A further type of medialess milling which may be employed in the present invention is high pressure dispersion milling in which dispersions are pressurized to 10,000-50,000 psi. At this point, the pressure is rapidly released. This release in pressure causes cavitation and grinding. Particles of 0.5 to 1 micron are typically produced via this method.

In the process known as media milling, balls, pebbles or other media such as sand are added in with material to be ground in order to reduce particle size. The collisions of the media with material to be ground results in the fracture of the large particles into smaller such particles. Using media milling, particles can be milled to average sizes of 0.1 micron with relative ease. Through control of the grinding time and force with which the material is ground, virtually any particle size can be obtained. Media mill can be used with or without any liquid additives, although water or other solvents are commonly used to produce the finest particle.

Until recently, the actual quantitation of milling in terms of size distribution and the effects of process variables have been complicated. Mathematical models predicting the size and size distribution of milled particles have been developed and deemed valid by the demonstrated agreement with laboratory results. For jet milling for example, it is now known how variables such as feed rate, angle of inlet nozzle and air flow rate affect the process of micronization. This allows for much greater control over resultant particle size with narrower size distribution.

Therefore, one aspect of the present invention includes utilization of one or more micronization or fine-milling techniques in order to provide a rapid release formulation comprising amino acids. Utilization of one or more of the micronization and fine-milling techniques increases rate of dissolution and ensures an increased number of molecules provided to gastric juices, thereby improving bioavailability. Since more molecules will be available in a readily absorbable state, and thus bio-absorption prior to excretion from an individual's body, the method of the present invention thereby improves bioavailability.

Moreover, with respect to the small particle size of fine-milled products, there may be an improved taste, flavor-enhancement, texture and palatably of compositions comprising poorly-soluble compounds which have been fine-milled owing to the improved rate of dissolution.

### General Formulation

Oral formulations of amino acids are disclosed which are comprised of one or more amino acids and one or more excipients. The formulation of amino acids and excipients is designed to obtain a desired result, e.g. enhanced skeletal muscle anabolism. The desired results are obtained by increasing the rate of bioavailability, and increasing the immediacy of dissolution and absorption of a therapeutic level of amino acid as compared to regular formulations.

The formulation of the invention is preferably an oral dosage formulation that may be in any suitable oral form including tablets, capsules, caplets, suspensions, powders, etc. The dosage may be of any desired size in terms of the active ingredient.

The amount an individual will need to obtain an optimum therapeutical effect will vary with a number of factors known to those skilled in the art, e.g., the size, age, weight, sex and condition of the individual. A particularly preferred formulation will provide a rapid release of the amino acid and increased rate of bioavailability.

### Rapid Release Technology

It is herein understood that, due to the relationship between solubility and dissolution, the amount of a substance in solution at any given time is dependent upon both dissolution and solubility. It is also understood that before an orally consumed active ingredient is bioavailable, it must first be in solution. Furthermore, it is understood by way of extension that increasing the rate of dissolution of a given substance may act to reduce the time to dissolution of a given solute or substance in a given solvent. Thus, increasing the rate of dissolution of a substance will increase the amount of said substance in solution at earlier points in time, thus increasing the rate of bioavailability of said substance at earlier times upon oral administration.

As it relates to this invention, micronization techniques are employed to increase the rate of bioavailability of said supplemental dietary ingredients. The increase in the rate of bioavailability will allow enhanced and quicker compound transfer to the systemic parts of the body following ingestion, and thus increase efficacy of the compositions comprising such supplemental dietary ingredients.

Micronization is a technique, which has been used as a method of sizing solid compounds to fine powders. Following a micronization process, said poorly soluble compounds are transformed into fine powders, which can then be transformed into suitable, stable, and individual-compliant dosage forms. These forms, for the purposes of the present invention are derived for oral administration.

Micronization-technique treated poorly-soluble compounds or dietary supplement ingredients are preferred in the present invention in that they offer an advantage over larger forms of poorly soluble compounds—following micronization, compounds have higher surface area to volume ratio. This provides for, as compared to physically coarse compounds, an ultrafine micronized powder that has a significantly increased total surface area. Mathematically, cross-sectional surface area increases with the square of the radius, while volume increases with the cube of the radius. Therefore, as a particle becomes smaller, the volume of the particle decreases at a faster rate than the surface area leading to an increase in the ratio of surface area to volume. By way of theoretical calculations, decreasing the size of a particle can increase its rate of dissolution via increasing the surface area to volume ratio. In the case of solubility, this increase in relative surface area allows for greater interaction with solvent.

For example, if we consider a particle with a diameter of 1 cm it will have a V of 0.523 cm³ and a SA of 3.14 cm². If such a theoretical particle is milled to one-tenth the diameter, each milled particle will have V of 0.000523 cm³ and SA of 0.0314 cm². The volume occupied by one of the larger particles may contain up to approximately 1000 of the milled particles. This corresponds to an approximate total surface area of 31.4 cm², an increase of about 10-fold. This increase in surface area should result in an increase in solubility or dissolution.

In a preferred embodiment of the present invention, one or more amino acids are provided in an oral dosage formulation that may be in any suitable oral form including tablets, capsules, caplets, suspensions, powders, etc., wherein one or more amino acids are provided in both a milled form and an un-milled form. In such an embodiment, multiphasic dissolution of the one or more amino acids is achieved wherein the milled portion of the one or more amino acids is released or dissolved before the un-milled portion.

In an additional embodiment of the present invention, the amino acids are provided in an oral dosage formulation that may be in any suitable oral form including tablets, capsules, caplets, suspensions, powders, etc. together with excipients commonly known in the art to alter the release of the amino acids. Such excipients include those known to result in rapid or immediate release, as well as those known to result in delayed or time-release.

### EXAMPLES

In order to determine the effect of fine-milling on the dissolution rate, initial testing was performed to examine the rate of dissolution of common supplemental dietary ingredients. For the purposes of this disclosure, the term regular” as used herein makes reference to non-fine-milled particles.

Experiments relating to fine-milling and bioavailability were undertaken by the inventors. Outlines and the result of said experiments are given below.

### Testing and Experimentation

The method employed was based on the United States Pharmacopeia (USP) Apparatus 2 Dissolution Testing, which employs a USP paddle apparatus. Essentially, a rotating paddle powered by a motor is placed inside a liquid-containing vessel to promote the dissolution of solid oral dosage forms placed in the vessel. The vessel contains a known volume of liquid held at known constant temperature; the paddle rotates at known and constant rotations per minute (rpm). Aliquots are removed at specific times to test for the amount of specific active agent present.

Tablets were produced containing either a fine-milled amino acid or an equivalent amount of the same non-fine-milled amino acid, each tablet type containing the same excipients and similarly produced.

### Example 1

### Leucine

The dissolution testing volume consisted of 500 mL of water held at a constant temperature of 37° C. The paddle rotation was maintained at a constant speed of 75 rpm. Tablets containing either regular Leucine or fine-milled Leucine were separately placed into the apparatus and allowed to dissolve. Aliquots were removed at 5, 10, 15, 30, 45 and 60 minutes. Leucine present in each aliquot was detected by High Performance Liquid Chromatography (HPLC). The relative amount of Leucine present in solution as a function of time is shown in FIG. 1.

From the results shown in FIG. 1, more milled Leucine compared to regular Leucine was determined to be in solution after 15 minutes. The greater amount of milled Leucine compared to regular Leucine in solution was maintained at all subsequently examined time points. Before 15 minutes, at 5 and 10 minutes, there was slightly more regular Leucine in solution compared to milled Leucine. The difference at these early time points was much less than at the later times.

These data indicate that a consistently greater amount of Leucine is available for absorption from tablets containing milled Leucine than from tablets containing regular Leucine at equivalent time points at or after 15 minutes of exposure to agitation and solvent.

### Example 2

### Arginine

The dissolution testing volume consisted of 500 mL of water held at a constant temperature of 37° C. The paddle rotation was maintained at a constant speed of 75 rpm. Tablets containing either regular Arginine or fine-milled Arginine were separately placed into the apparatus and allowed to dissolve. Aliquots were removed at 5, 10, 15, 30, 45 and 60 minutes. Arginine present in each aliquot was detected by High Performance Liquid Chromatography (HPLC). The relative amount of Arginine present in solution as a function of time is shown in FIG. 2.

From the results shown in FIG. 2, considerably more milled Arginine compared to regular Arginine was determined to be in solution at all examined time points. The greater amount of milled Arginine compared to regular Arginine in solution was maintained at a relatively consistent level at all examined time points.

These data indicate that a consistently greater amount of Arginine is available for absorption from tablets containing milled Arginine than from tablets containing regular Arginine as early as 5 minutes after exposure to agitation and solvent.

### Example 3

### Phenylalanine

The dissolution testing volume consisted of 500 mL of water held at a constant temperature of 37° C. The paddle rotation was maintained at a constant speed of 75 rpm. Tablets containing either regular Phenylalanine or fine-milled Phenylalanine were separately placed into the apparatus and allowed to dissolve. Aliquots were removed at 5, 10, 15, 30, 45 and 60 minutes. Phenylalanine present in each aliquot was detected by Capillary Electrophoresis (CE). The relative amount of Phenylalanine present in solution as a function of time is shown in FIG. 3.

From the results shown in FIG. 3, considerably more milled Phenylalanine compared to regular Phenylalanine was determined to be in solution at all examined time points. The greater amount of milled Phenylalanine compared to regular Phenylalanine in solution was lessened at later time points due to an increasing amount of regular Phenylalanine in solution.

These data indicate that a consistently greater amount of Phenylalanine is available for absorption from tablets containing milled Phenylalanine than from tablets containing regular Phenylalanine as early as 5 minutes after exposure to agitation and solvent. The amount of Phenylalanine in solution from tablets containing milled Phenylalanine was essentially equivalent at all examined time points. The amount of Phenylalanine in solution from tablets containing regular Phenylalanine, however, was increased at each time relative to the earlier time point. This suggests that at times greater than 60 minutes the amount of Phenylalanine in solution may be similar for both milled and regular Phenylalanine-containing tablets.

### Example 4

### Hydroxycitric Acid

For comparison, Hydroxycitric acid, a substance unrelated to amino acids was also examined. The dissolution testing volume consisted of 500 mL of 0.1 N HCl held at a constant temperature of 37° C. The paddle rotation was maintained at a constant speed of 75 rpm. Tablets containing either regular Hydroxycitric acid or fine-milled Hydroxycitric acid were separately placed into the apparatus and allowed to dissolve. Aliquots were removed at 5, 10, 15, 30, 45 and 60 minutes. Hydroxycitric acid present in each aliquot was detected by High Performance Liquid Chromatography (HPLC). The relative amount of Hydroxycitric acid present in solution as a function of time is shown in FIG. 5.

From the results shown in FIG. 4, considerably more regular Hydroxycitric acid compared to milled Hydroxycitric acid was determined to be in solution at 5, 10 and 15 minutes. For the remainder of the examined time points, the amount of regular Hydroxycitric acid in solution was consistently slightly higher than was milled Hydroxycitric acid.

These data suggest that milling of Hydroxycitric acid may not afford the same benefit shown for the tested amino acids. This finding was surprising, considering the mechanism by which nano-milling is proposed to work.

### Discussion

The results of the experiments demonstrate that tablets containing milled Leucine, Arginine or Phenylalanine cause a more rapid tablet dissolution compared to tablets containing regular respective amino acids. This translates into more of the given amino acid present in solution available for absorption. Given that a typical time for gastric emptying is about 40 minutes, tablets containing milled Leucine, Arginine or Phenylalanine will provide an increased immediacy of absorption of the particular amino acid and enhanced bioavailability compared to tablets containing regular Leucine, Arginine or Phenylalanine. The same improvement was not demonstrated for Hydroxycitric acid (an unrelated substance), suggesting that the efficacy of any specific milled ingredient should be experimentally demonstrated.

### Example 5

### Amino Acid Caplet

The following composition is to be provided as a dietary supplement for consumption by humans in the form of a caplet for enhancing skeletal muscle growth and repair. One serving of the composition comprises 5 caplets comprising the following:
about 2 g Leucine, about 0.1 g of Leucine (milled), about 1.2 g Microcrystalline cellulose, about 0.5 g Anhydrous dicalcium phosphate, about 0.3 g Polyvinylpolypyrrolidone, about 0.25 g Croscarmellose sodium, and about 0.2 g Stearic acid.

One serving of the composition is to be taken once daily, preferably after exercise.

### Example 6

### Amino Acid Tablet

The following composition is to be provided as a dietary supplement for consumption by humans in the form of a tablet for enhancing vasodilation of blood vessels. One serving of the composition comprises 3 tablets comprising the following:

about 2.9 g Arginine, about 0.1 g Arginine (milled), about 0.75 g Microcrystalline cellulose, about 0.31 g Hydroxypropylcellulose, about 0.05 g Crospovidone and about 0.1 g Croscarmellose sodium.

One serving of the composition is to be taken twice daily, preferably at least once immediately prior to exercise.

What is claimed:

1. A rapid release oral dosage formulation, comprising at least one amino acid; wherein at least a portion of the amino acid has been milled in a manner so as to increase the immediacy of dissolution and absorption of said amino acid in a mammal as compared to regular formulations.

2. The rapid release oral dosage formulation of claim 1, wherein the amino acid is Leucine or derivatives of Leucine.

3. The rapid release oral dosage formulation of claim 1, wherein the amino acid is Arginine or derivatives of Arginine.

4. The rapid release oral dosage formulation of claim 1, wherein the amino acid is Phenylalanine or derivatives of Phenylalanine.

5. The rapid release oral dosage formulation of claim 1, wherein the dosage format is one of a tablet, a capsule or a caplet.

6. A rapid release oral dosage formulation, comprising: about 0.9% to about 15% by mass of a milled amino acid ; wherein the formulation is characterized by rapid releasing the amino acid in a manner so as to increase the immediacy of absorption of said amino acid in said mammal as compared to regular formulations not containing milled amino acids.

7. The rapid release oral dosage formulation of claim 6, wherein the amino acid is Leucine or derivatives of Leucine.

8. The rapid release oral dosage formulation of claim 7, wherein the amino acid is Arginine or derivatives of Arginine.

9. The rapid release oral dosage formulation of claim 7, wherein the amino acid is Phenylalanine or derivatives of Phenylalanine

10. A method for increasing the immediacy of dissolution and absorption of an amino acid in a mammal comprising administering to the mammal the formulation of claim 1, wherein the amino acid is selected from the group consisting of Leucine, Arginine, Phenylalanine, or derivatives thereof.
